SEQUENCE LISTING (2 sequences)

SEQ ID NO: 1 - DNA, 1740 bp, Gluconobacter oxydans
atgaaccccacaacgctgcttcgcaccagcgcggccgtgctattgcttaccgcgcccgccgcattcgcgcaggtaaccccgattaccgatgaactgctggcgaacccgcccgctggtgaatggattaactacggccgcaaccaagaaaactatcgccactcgcccctgacccagatcactgccgacaacgttggtcagttgcaactggtctgggcccgcgggatggaggcgggggccgtacaggtcacgccgatgatccatgatggcgtgatgtatctggcaaaccccggtgatgtgatccaggcgctggatgcgcaaacaggcgatctgatctgggaacaccgccgccaactgcccgccgtcgccacgctaaacgcccaaggcgaccgcaagcgcggcgtcgccctttacggcacgagcctctatttcagctcatgggacaaccatctgatcgcgctggatatggagacgggccaggtcgtattcgatgtcgaacgtggatcgggcgaagacggcttgaccagtaacaccacggggccgattgtcgccaatggcgtcatcgtcgcgggttccacctgccaatattcgccctatggatgctttatctcggggcacgattccgcgacgggtgaggagctgtggcgcaaccactttatcccgcagccgggcgaagagggtgacgagacttggggcaatgatttcgaggcgcgctggatgaccggcgtctggggtcagatcacctatgatcccgtgacgaaccttgtgttctatggctcgaccggcgtgggcccagcgtccgaaacccagcgcggcacgccgggcggcacgctgtatggcaccaacacccgctttgcggtgcgtcccgacacgggcgagattgtctggcgtcaccagaccctgccgcgcgacaactgggaccaagaatgcacgttcgagatgatggtcgccaacgtcgatgtgcaaccctcggccgagatggagggtctgcgcgccatcaaccccaatgcggcgacgggcgagcgccgtgtgctgacgggtgcgccttgcaagaccggcacgatgtggtcgtttgatgcggcctcgggcgaattcctgtgggcgcgtgataccaactacaccaatatgatcgcctcgatcgacgagaccggccttgtgacggtgaacgaggatgcggtgctgaaagagctggacgttgaatatgacgtctgcccgaccttcctgggtgggcgcgactggtcgtcagccgcactgaacccggacaccggcatttacttcttgccgctgaacaatgcctgctacgatattatggccgttgatcaagagtttagcgcgctcgacgtctataacaccagcgcgaccgcaaaactcgcgccgggctttgaaaatatgggccgcatcgacgcgattgatatcagcaccgggcgcaccttgtggtcggcggagcgccctgcggcgaactactcgcccgttttgtcgacggcaggcggtgtggtgttcaacggcgggaccgaccgctatttccgtgccctcagccaggaaaccggcgagactttgtggcaggcccgtcttgcgacggtcgcgacggggcaggcgatcagctacgagttggacggcgtgcaatatatcgccatcggtgcgggcggtctgacctatggcacgcaattgaacgcgccgctggccgaggcaatcgattcgacctcggtcggtaatgcgatctatgtctttgcactgccgcagtaa

SEQ ID NO: 2 - protein, 579 aa, Gluconobacter oxydans
MNPTTLLRTSAAVLLLTAPAAFAQVTPITDELLANPPAGEWINYGRNQENYRHSPLTQITADNVGQLQLVWARGMEAGAVQVTPMIHDGVMYLANPGDVIQALDAQTGDLIWEHRRQLPAVATLNAQGDRKRGVALYGTSLYFSSWDNHLIALDMETGQVVFDVERGSGEDGLTSNTTGPIVANGVIVAGSTCQYSPYGCFISGHDSATGEELWRNHFIPQPGEEGDETWGNDFEARWMTGVWGQITYDPVTNLVFYGSTGVGPASETQRGTPGGTLYGTNTRFAVRPDTGEIVWRHQTLPRDNWDQECTFEMMVANVDVQPSAEMEGLRAINPNAATGERRVLTGAPCKTGTMWSFDAASGEFLWARDTNYTNMIASIDETGLVTVNEDAVLKELDVEYDVCPTFLGGRDWSSAALNPDTGIYFLPLNNACYDIMAVDQEFSALDVYNTSATAKLAPGFENMGRIDAIDISTGRTLWSAERPAANYSPVLSTAGGVVFNGGTDRYFRALSQETGETLWQARLATVATGQAISYELDGVQYIAIGAGGLTYGTQLNAPLAEAIDSTSVGNAIYVFALPQ

(19) United States
(12) Patent Application Publication
(10) Pub. No.: US 2005/0244940 A1
(43) Pub. Date: Nov. 3, 2005

(54) Process for producing l-ascorbic acid

(75) Inventors: Tatsuo Hoshino, Kamakura-shi (JP); Masako Shinjoh, Basel (CH)

Correspondence Address: Stephen M Haracz;Bryan Cave, 1290 Avenue of the Americas, New York, NY, 10104, US

(73) Assignee: DSM IP Assets B.V., 6411 TE Heerlen (NL)

(21) Appl. No.: 10/528,673

(22) PCT Filed: Sep. 22, 2003
(86) PCT No.: PCT/EP03/10489

§ 371 Date: Mar. 23, 2005

(30) Foreign Application Priority Data
Sep. 27, 2002 (EP) 02021602.4

(51) Int. Cl. C12P 17/06; C12N 9/02

(52) U.S. Cl. 435/125; 435/189

(57) ABSTRACT

The present invention provides the use of Enzyme B of G. oxydans DSM 4025, as disclosed in EP 0 832 974 A2, in a process for producing L-ascorbic acid from L-gulose, L-galactose, L-idose or L-talose, or from L-gulono-1,4-lactone (and its acid form, L-gulonic acid) and from L-galactono-1,4-lactone (and its acid form, L-galactonic acid).

The present invention relates to the use of Enzyme B of Gluconobacter oxydans DSM 4025 as disclosed in EP 0 832 974 A2 in a process for producing L-ascorbic acid.

Feasibility studies on the biotechnological synthesis of L-ascorbic acid (vitamin C) were performed for many years since the “Reichstein method” was established in 1934. The microorganisms Gluconobacter oxydans DSM 4025 or Escherichia coli carrying the D-arabinono-1,4-lactone oxidase gene of Saccharomyces cerevisine, Candida albicans and Saccharomyces cerevisiae oxidize L-galactono-1,4-lactone to vitamin C. Saccharomyces cerevisiae and Candida albicans possess a D-arabinose dehydrogenase catalyzing the production of D-arabinono-1,4-lactone and L-galactono-1,4-lactone from D-arabinose and L-galactose, respectively. However, there were no reports describing the possibility of biological vitamin C production from another L-hexose as intermediate, that is, L-idose, L-gulose, and L-talose, with a configuration corresponding to that of vitamin C (C4 and C5 positions).

The present invention provides the use of Enzyme B of G. oxydans DSM 4025, as disclosed in EP 0 832 974 A2, in a process for producing L-ascorbic acid from L-gulose, L-galactose, L-idose or L-talose, or from L-gulono-1,4-lactone (and its acid form, L-gulonic acid) and from L-galactono-1,4-lactone (and its acid form, L-galactonic acid).

The present invention also provides the use of Enzyme B of G. oxydans DSM 4025, as disclosed in EP 0 832 974 A2, in a process for producing L-gulono-1,4-lactone or L-galactono-1,4-lactone, or their acid forms L-gulonic acid or L-galactonic acid from L-gulose or L-galactose, respectively.

EP 0 832 974 A2, which is incorporated by reference, discloses Enzyme B of G. oxydans DSM 4025 having the following physico-chemical properties:
(a) molecular weight of about 60,000 Da on SDS-PAGE;
(b) substrate specificity for primary and secondary alcohols and aldehydes;
(c) pH-stability at pH of about 6 to about 9;
(d) pH-optimum at pH of about 8.0; and
(e) inhibited by Cu²⁺, Zn²⁺, Mn²⁺, Fe², and Fe³⁺.

Furthermore, the amino acid sequences as well as the nucleotide sequences of Enzyme B are disclosed in EP 0 832 974 A2 and represented by SEQ ID NO: 2 and SEQ ID NO: 1 of the present sequence listing. It is known from EP 0 832 974 A2 that Enzyme B is able to convert D-glucose, D-sorbitol, L-sorbosone, D-mannitol, L-idose, glycerol, D-gluconic acid, D-mannoic acid to D-gluconate, L-sorbose, 2-KGA, D-fructose, L-idonic acid, dihydroxyacetone, 5-keto-D-gluconic acid, and 5-keto-D-mannoic acid, respectively.

The present invention is related to a process for the production of L-ascorbic acid with an enzyme having the amino acid sequence of SEQ ID NO: 2 or an amino acid sequence that is 90% identical thereto, with the activity to produce L-ascorbic acid, whereby L-ascorbic acid is produced from a substrate which is selected from the group consisting of L-gulose, L-galactose, L-idose, and L-talose.

L-Hexoses like L-gulose, L-galactose, L-idose, and L-talose are rare sugars which are basically produced by chemical methods and are commercially high-cost compounds. However, biological preparations for L-gulose and L-galactose have been recently reported. L-Gulose production from D-sorbitol by Enzyme A of G. oxydans DSM 4025 was reported in EP 0 832 974 A2. L-Gulose production from L-sorbose by L-ribose isomerase was disclosed in U.S. Pat. No. 6,037,153. L-Galactose production from L-sorbose is reported by Izumori et al. (2001 Annual Meeting of the Society for Bioscience and Bioengineering, Japan). In this process they combined two enzymatic processes consisting of “L-sorbose to L-tagatose” reaction with L-tagatose epimerase of Pseudomonas cichorii ST-24 strain (U.S. Pat. No. 5,811,271) and “L-tagatose to L-galactose” reaction with D-arabinose isomerase of Bacillus stearothermophilus 14a strain. L-Gulono-1,4-lactone may be prepared from D-glucose.

In another aspect the present invention provides a process for
(a) producing
(i) L-ascorbic acid from L-gulose or L-galactose by an enzyme which comprises contacting L-gulose or L-galactose with an enzyme having an amino acid sequence of SEQ ID NO: 2 or an amino acid sequence that is 90% identical thereto, or a portion thereof, with the activity to produce L-ascorbic acid from L-gulose and L-galactose or a functional equivalent thereof, in a reaction mixture,
(ii) L-gulono-1,4-lactone from L-gulose, or L-galactono-1,4-lactone from L-galactose by an enzyme which comprises contacting L-gulose or L-galactose with an enzyme having an amino acid sequence of SEQ ID NO:2 or an amino acid sequence that is 90% identical thereto, or a portion thereof, with the activity to produce L-gulono-1,4-lactone from L-gulose and L-galactono-1,4-lactone from L-galactose, or a functional equivalent thereof, in a reaction mixture, or
(iii) L-ascorbic acid from L-gulono-1,4-lactone or from L-galactono-1,4-lactone by an enzyme which comprises contacting L-gulono-1,4-lactone or from L-galactono-1,4-lactone with the enzyme having an amino acid sequence of SEQ ID NO: 2 or an amino acid sequence that is 90% identical thereto, or a portion thereof, with the activity to produce L-ascorbic acid from L-gulono-1,4-lactone and from L-galactono-1,4-lactone, or a functional equivalent thereof, in a reaction mixture,
and
(b) isolating L-ascorbic acid, L-gulono-1,4-lactone or L-galactono-1,4-lactone from the reaction mixture.

It is one embodiment of the present invention to provide a process for the production of L-ascorbic acid with an enzyme having the amino acid sequence of SEQ ID NO: 2 or an amino acid sequence that is 90% identical thereto, with the activity to produce L-ascorbic acid, whereby L-ascorbic acid is produced from a substrate which is selected from the group consisting of L-gulono-1,4-lactone, L-gulonic acid, L-galactono-1,4-lactone, L-galactonic acid, L-idono-1,4-lactone, L-idonic acid, L-talono-1,4-lactone, and L-talonic acid.

In a further embodiment, the present invention covers a process for the production of L-gulono-1,4-lactone or L-gulonic acid with an enzyme having the amino acid sequence of SEQ ID NO: 2 or an amino acid sequence that is 90% identical thereto, with the activity to produce L-gulono-1,4-lactone or L-gulonic acid, whereby L-gulono-1,4-lactone or L-gulonic acid is produced from L-gulose.

Furthermore, it is an aspect of the present invention to provide a process for the production of L-galactono-1,4-lactone or L-galactonic acid with an enzyme having the amino acid sequence of SEQ ID NO: 2 or an amino acid sequence that is 90% identical thereto, with the activity to produce L-galactono-1,4-lactone or L-galactonic acid, whereby L-galactono-1,4-lactone or L-galactonic acid is produced from L-galactose.

A process according to the present invention as described above comprises (a) contacting the enzyme with the respective substrate and (b) isolating the respective product from the reaction mixture. In case of vitamin C as product, the substrate is selected from the group consisting of L-gulose, L-galactose, L-idose, L-talose, L-gulono-1,4-lactone, L-gulonic acid, L-galactono-1,4-lactone, L-galactonic acid, L-idono-1,4-lactone, L-idonic acid, L-talono-1,4-lactone, and L-talonic acid. In case of L-gulono-1,4-lactone or L-gulonic acid as product, the substrate is L-gulose. In case of L-galactono-1,4-lactone or L-galactonic acid as product, the substrate is L-galactose.

In the present invention, a functional equivalent of the enzyme can be made either by chemical peptide synthesis known in the art or by recombinant means on the basis of the DNA sequences as disclosed herein by methods known in the state of the art. Amino acid exchanges in proteins and peptides which do not generally alter the activity of such molecules are known in the state of the art. The most commonly occurring exchanges are: Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, Asp/Gly as well as these in reverse.

Furthermore such functional equivalent of the enzyme includes an amino acid sequence encoded by a DNA sequence of SEQ ID NO: 1 as disclosed, e.g., in the sequence listing as well as the complementary strand, or those which include the sequences, DNA sequences which hybridize under standard conditions with such sequences or fragments thereof and DNA sequences, which because of the degeneration of the genetic code, do not hybridize under standard conditions with such sequences but which code for polypeptides having exactly the same amino acid sequence, wherein the functional equivalent has the enzymatic activity of producing
(i) L-ascorbic acid from L-gulose and from L-galactose,
(ii) L-gulono-1,4-lactone (and its acid form, L-gulonic acid) from L-gulose and L-galactono-1,4-lactone (and its acid form, L-galactonic acid) from L-galactose, or
(iii) L-ascorbic acid from L-gulono-1,4-lactone (and its acid form, L-gulonic acid) and from L-galactono-1,4-lactone (and its acid form, L-galactonic acid).

“Standard conditions” for hybridization mean in this context the conditions which are generally used by a man skilled in the art to detect specific hybridization signals, or preferably so called stringent hybridization and non-stringent washing conditions or more preferably so called stringent hybridization and stringent washing conditions a man skilled in the art is familiar with. Furthermore, DNA sequences which can be made by the polymerase chain reaction by using primers designed on the basis of the DNA sequences disclosed herein by methods known in the art are also an object of the present invention. It is understood that the DNA sequences of the present invention can also be made synthetically as described, e.g. in EP 747 483 A2.

A mutant of the gene can be prepared by treating the gene or a microorganism carrying the gene with a mutagen such as ultraviolet irradiation, X-ray irradiation, γ-ray irradiation or contact with a nitrous acid, N-methyl-N′-nitro-N-nitrosoguanidine (NTG), or other suitable mutagens, or isolating a colony or clone occurring by spontaneous mutation or by standard methods of in vitro mutagenesis known in the art. Many of these methods have been described in various publications.

As used herein, a “mutant” is any gene that encodes a non-native polynucleotide sequence or a polynucleotide sequence that has been altered from its native form (such as, e.g., by rearrangement or deletion or substitution of from 1-100, preferably 20-50, more preferably less than 10 nucleotides). As noted above, such a non-native sequence may be obtained by random mutagenesis, chemical mutagenesis, spontaneous mutation, UV-irradiation, PCR-prone error generation, site-directed mutagenesis, and the like. Preferably, the mutation results in the expression of a polypeptide having the increased production or improved activity compared to a non-mutant parental polypeptide using the assay procedures set forth in the Examples. Methods for generating, screening for, and identifying such mutant cells are well known in the art.

A specific and preferred G. oxydans strain as a donor of a DNA sequence encoding a polypeptide has been deposited at the Deutsche Sammlung von Mikroorganismen und Zellkulturen (DSMZ) in Göttingen (Germany) under DSM No. 4025, which was done under the stipulations of the Budapest Treaty on Mar. 17, 1987. A biologically and/or taxonomically homogeneous culture of a microorganism having the identifying characteristics G. oxydans DSM 4025 can also be a donor of the DNA sequence.

A subculture of G. oxydans DSM 4025 has been deposited in the Agency of Industrial Science and Technology, Fermentation Research Institute, Japan, under the deposit No.: FERM BP-3812. EP 0 278 447 B1 discloses the characteristics of this strain.

By using the information of the so determined nucleotide sequence (in consideration of the codon usage) a gene encoding evolutionally divergent enzyme having the activity of producing (i) L-ascorbic acid from L-gulose and L-galactose, (ii) L-gulono-1,4-lactone (and its acid form, L-gulonic acid) from L-gulose and L-galactono-1,4-lactone (and its acid form, L-galactonic acid) from L-galactose, or (iii) L-ascorbic acid from L-gulono-1,4-lactone (and its acid form, L-gulonic acid) and from L-galactono-1,4-lactone (and its acid form, L-galactonic acid), vitamin C forming-activity from L-gulose or L-galactose, can be isolated from a different organism by colony- or Southern-hybridization with a probe synthesized according to the amino acid sequence deduced from said nucleotide sequence or by the polymerase chain reaction with primers also synthesized according to said information, if necessary.

Furthermore, a preferred host microorganism for constructing a recombinant microorganism carrying Enzyme B gene of G. oxydans DSM 4025 and its functional equivalent or mutant defined above may be Escherichia coli, Pseudomonas putida and G. oxydans DSM 4025 and their biologically and/or taxonomically homogeneous culture or mutant.

To construct a recombinant microorganism carrying the gene for Enzyme B and its functional equivalent or mutant on a recombinant expression vector or on a chromosomal DNA of a host microorganism, various gene transfer methods including transformation, transduction, conjugal mating, and electroporation can be used. The method for constructing a recombinant organism may be selected from the methods well known in the field of molecular biology. Usual transformation systems can be used for Escherichia coli, and Pseudomonas. A transduction system can also be used for Escherichia coli. Conjugal mating systems can be widely used in Gram-positive and Gram-negative bacteria including E. coli, Pseudomonas putida and G. oxydans. The conjugation can occur in liquid media or on a solid surface. The preferred recipient is selected from E. coli, Pseudomonas putida and G. oxydans which can produce active Enzyme B with a suitable recombinant expression vector. To the recipient for conjugal mating, a selective marker is usually added; for example, resistance against nalidixic acid or rifampicin is usually selected.

The microorganisms provided in the present invention may be cultured in an aqueous medium supplemented with appropriate nutrients under aerobic conditions. The cultivation may be conducted at a pH between about 1.0 and about 9.0, preferably between about 2.0 and about 8.0. While the cultivation period varies depending upon pH, temperature and nutrient medium used, usually 2 to 5 days will bring about favorable results. A suitable temperature range for carrying out the cultivation is from about 13° C. to about 45° C., preferably from about 18° C to about 42° C.

Thus, the process for the production of vitamin C of the present invention as described above is conducted for 1 to 120 h at a pH of about 1 to about 9 and at a temperature of about 13° C. to about 45° C. Preferably, the process is conducted at a pH of about 2 to about 8 and at a temperature of about 13° C. to about 45° C. These conditions are also applied to the process for the production of L-gulono-1,4-lactone, L-gulonic acid, L-galactono-1,4-lactone or L-galactonic acid, as further embodiments of the present invention.

It is usually required that the culture medium contains such nutrients as assimilable carbon sources, digestible nitrogen sources and inorganic substances, vitamins, trace elements and other growth promoting factors. Examples for assimilable carbon sources include glycerol, D-glucose, D-mannitol, D-fructose, D-arabitol, D-sorbitol and L-sorbose.

Various organic or inorganic substances may also be used as nitrogen sources, such as yeast extract, meat extract, peptone, casein, corn steep liquor, urea, amino acids, nitrates, ammonium salts and the like. As inorganic substances, magnesium sulfate, potassium phosphate, ferrous and ferric chlorides, calcium carbonate and the like may be used.

As used herein, L-ascorbic acid means that the substance exists either as a free acid form or as a salt form such as Na-salt, K-salt, or hemicalcium-salt, such as calcium L-ascorbate dihydrate. Moreover, concentration of L-ascorbic acid is described as the free acid form unless otherwise stated.

As used herein, L-gulono-1,4-lactone and L-galactono-1,4-lactone mean that the substances exist as their lactone forms and/or their acid forms, both of which exist in an equilibrium state under various physico-chemical conditions.

As used herein the phrase “standard conditions for hybridization” means conditions which are generally used by a person skilled in the art to detect specific hybridization signals, or preferably, so called stringent hybridization and non-stringent washing conditions, or more preferably, so called moderately stringent conditions, or even more preferably, so called stringent hybridization and stringent washing conditions which a person skilled in the art is familiar with.

For example, any combination of the following hybridization and wash conditions may be used, as appropriate:
High Stringency Hybridization: 6×SSC, 0.5% SDS, 100 μg/ml denatured salmon sperm DNA, 50% formamide, incubate overnight at 42° C. with gentle rocking.
High Stringency Wash: 1 wash in 2×SSC, 0.5% SDS at room temperature for 15 minutes, followed by another wash in 0.1×SSC, 0.5% SDS at room temperature for 15 minutes.
Low Stringency Hybridization: 6×SSC, 0.5% SDS, 100 μg/ml denatured salmon sperm DNA, 50% formamide, incubate overnight at 37° C. with gentle rocking.
Low Stringency Wash: 1 wash in 0.1×SSC, 0.5% SDS at room temperature for 15 minutes.

Moderately stringent conditions may be obtained by varying the temperature at which the hybridization reaction occurs and/or the wash conditions as set forth above.

The concentration of the substrates including L-aldose and L-aldonolactone in a reaction mixture can vary depending on other reaction conditions, but, in general, may be between about 1 g/l and about 300 g/l, preferably between about 10 g/l and about 200 g/l.

The reaction can be conducted aerobically.

For the reaction, the enzyme can be used in any form, including, but not limited to, enzyme solution, immobilized enzyme, intact cells, and immobilized cells may be used.

After the reaction, L-ascorbic acid or L-aldonolactone may be recovered from the reaction mixture by the combination of various kinds of chromatography, for example, thin layer chromatography, adsorption chromatography, ion-exchange chromatography, gel filtration chromatography or high performance liquid chromatography. L-Aldonolactone as a reaction product can also be used as a substrate for a further reaction as it is in the reaction mixture of this invention without purification.

The following examples are provided to further illustrate the process of the present invention. These examples are illustrative only and are not intended to limit the scope of the invention in any way.

### EXAMPLE 1

### Production of L-gulono-1,4-lactone/L-gulonic Acid from L-gulose by Escherichia coli JM109 Carrying the Enzyme B Gene

The gene encoding Enzyme B was cloned and subcloned from pSS103R into a vector pTrcMalE to construct pTrcMalE-EnzB as described in EP 0 832 974 A2. Escherichia coli JM109 carrying pTrcMalE-EnzB was grown on 2 ml of LB medium with 100 μg/ml of ampicillin at 30° C. for 15 h and 100 μl of the resulting broth was transferred into fresh LB medium with 100 μg/ml of ampicillin and incubated at 30° C. for 4 h. Then, IPTG was added to the culture at a final concentration of 0.2 mM and the culture was further cultivated at 30° C. for 2 h. The control cultivation of E. coli JM109 carrying pTrcMalE-EnzB without IPTG addition was also performed. Escherichia coli JM109 was used as the control strain in the same manner as described above, cultivation with or without addition of IPTG. The cells from 8 ml of the culture were collected by centrifugation and suspended in 1 ml of distilled water. The resulting cell suspension was used for the reaction with 400 μl of reaction mixture consisting of 250 μl of the cell suspension, 1% substrate, 0.3% NaCl, 1% CaCO₃, 1 μg/ml of PQQ and 1 mM PMS and the mixture was incubated at room temperature for 16 h. The substrate used in this experiment was L-gulose. The amounts of L-gulono-1,4-lactone plus L-gulonic acid and vitamin C are summarized in Table 1.

TABLE 1

| | | HPLC | |
|---|---|---|---|
| | | | Vitamin C |
| Strain | IPTG | L-GuL + L-GuA (mM) | (mg/L) |
| JM109/pTrcMalE-EnzB | + | 8.4 | 42.5 |
| | − | 4.6 | 10.5 |
| JM109 | + | nd | nd |
| | − | nd | nd |

L-GuL: L-gulono-1,4-lactone;
L-GuA: L-gulonic acid;
nd: not detected

### EXAMPLE 2

### Production of vitamin C from L-gulono-1,4lactone/L-gulonic Acid by Escherichia coli JM109 Carrying the Enzyme B Gene

The experiment was performed as described in Example 1, except for the substrate used which was L-gulono-1,4-lactone in this Example. The amount of vitamin C is summarized in Table 2.

TABLE 2

| | | HPLC |
|---|---|---|
| Strain | IPTG | Vitamin C (mg/L) |
| JM109/pTrcMalE-EnzB | + | 1.4 |
| | − | 1.2 |
| JM109 | + | nd |
| | − | nd |

nd: not detected

### EXAMPLE 3

### Production of L-galactono-1,4-lactone/L-galactonic Acid from L-galactose by Escherichia coli JM109 Carrying the Enzyme B Gene

The experiment was performed as described in Example 1, except for the substrate used which was L-galactose in this Example. The amounts of L-galactono-1,4-lactone plus L-galactonic acid and vitamin C are summarized in Table 3.

TABLE 3

| | | HPLC | |
|---|---|---|---|
| | | | Vitamin C |
| Strain | IPTG | L-GaL + L-GaA (mM) | (mg/L) |

| | | | |
|---|---|---|---|
| JM109/pTrcMalE-EnzB | + | 6.2 | 2.7 |
| | − | 3.9 | 1.6 |
| JM109 | + | nd | nd |
| | − | nd | nd |

L-GaL: L-galactono-1,4-lactone;
L-GaA: L-galactonic acid;
nd: not detected

### EXAMPLE 4

### Production of Vitamin C from L-galactono-1,4-lactone/L-galactonic Acid by Escherichia coli JM109 Carrying the Enzyme B Gene

The experiment was performed as described in Example 1, except for the substrate used which was L-galactono-1,4-lactone in this Example. The amount of vitamin C is summarized in Table 4.

TABLE 4

| | | HPLC |
|---|---|---|
| Strain | IPTG | Vitamin C (mg/L) |
| JM109/pTrcMalE-EnzB | + | 4.7 |
| | − | 3.7 |
| JM109 | + | nd |
| | − | nd |

nd: not detected

1. A process for the production of L-ascorbic acid comprising:
(a) contacting an enzyme with a substrate which is selected from the group consisting of L-gulose, L-galactose, L-idose, and L-talose; and
(b) isolating L-ascorbic acid from the reaction mixture,
wherein said enzyme has the amino acid sequence of SEQ ID NO: 2 or an amino acid sequence that is 90% identical thereto with the activity to produce L-ascorbic acid.

2. A process for the production of L-ascorbic acid with an enzyme having the amino acid sequence of SEQ ID NO: 2 or an amino acid sequence that is 90% identical thereto, with the activity to produce L-ascorbic acid, whereby L-ascorbic acid is produced from a substrate which is selected from the group consisting of L-gulono-1,4-lactone, L-gulonic acid, L-galactono-1,4-lactone, L-galactonic acid, L-idono-1,4-lactone, L-idonic acid, L-talono-1,4-lactone, and L-talonic acid.

3. A process for the production of L-gulono-1,4-lactone or L-gulonic acid with an enzyme having the amino acid sequence of SEQ ID NO: 2 or an amino acid sequence that is 90% identical thereto, with the activity to produce L-gulono-1,4-lactone or L-gulonic acid, whereby L-gulono-1,4-lactone or L-gulonic acid is produced from L-gulose.

4. A process for the production of L-galactono-1,4-lactone or L-galactonic acid with an enzyme having the amino acid sequence of SEQ ID NO: 2 or an amino acid sequence that is 90% identical thereto, with the activity to produce L-galactono-1,4-lactone or L-galactonic acid, whereby L-galactono-1,4-lactone or L-galactonic acid is produced from L-galactose.

5. A process according to claim 2 comprising (a) contacting the enzyme with the substrate and (b) isolating the L-ascorbic acid from the reaction mixture.

6. A process according to claim 1, wherein the process is conducted for 1 to 120 h at a pH of about 1 to about 9 and at a temperature of about 13° C. to about 45° C.

7. A process according to claim 6, wherein the process is conducted at a pH of about 2 to about 8 and at a temperature of about 18° C. to about 42° C.

8. A process for producing L-ascorbic acid comprising contacting a substrate which is selected from the group consisting of L-gulose, L-galactose, L-idose, L-talose, L-gulono-1,4-lactone, L-gulonic acid, L-galactono-1,4-lactone, and L-galactonic acid with Enzyme B of G. oxydans DSM 4025 and isolating L-ascorbic acid from the reaction mixture; wherein Enzyme B has the following physico-chemical properties:
(a) molecular weight of about 60,000 Da on SDS-PAGE;
(b) substrate specificity for primary and secondary alcohols and aldehydes;
(c) pH-stability at pH of about 6 to about 9;
(d) pH-optimum at pH of about 8.0; and
(e) inhibited by Cu²⁺, Zn²⁺, Mn²⁺, Fe²⁺, and Fe³⁺.

9. A process for producing L-gulono-1,4-lactone or L-gulonic acid comprising contacting L-gulose with Enzyme B of G. oxydans DSM 4025 and isolating L-gulono-1.4-lactone or L-gulonic acid from the reaction mixture, wherein Enzyme B has the following physico-chemical properties:
(a) molecular weight of about 60,000 Da on SDS-PAGE;
(b) substrate specificity for primary and secondary alcohols and aldehydes;
(c) pH-stability at pH of about 6 to about 9;
(d) pH-optimum at pH of about 8.0; and
(e) inhibited by Cu²⁺, Zn²⁺, Mn²⁺, Fe²⁺, and Fe³⁺.

10. A process for producing L-galactono-1,4-lactone or galactonic acid comprising contacting L-galactose with Enzyme B of G. oxydans DSM 4025 and isolating L-galactono-1,4-lactone or galactonic acid from the reaction mixture, wherein Enzyme B has the following physico-chemical properties:
(a) molecular weight of about 60,000 Da on SDS-PAGE;
(b) substrate specificity for primary and secondary alcohols and aldehydes;
(c) pH-stability at pH of about 6 to about 9;
(d) pH-optimum at pH of about 8.0; and
(e) inhibited by Cu²⁺, Zn²⁺, Mn²⁺, Fe²⁺, and Fe³⁺.

11. A process according to claim 3 comprising (a) contacting the enzyme with the substrate and (b) isolating the L-gulono-1,4-lactone or L-gulonic acid from the reaction mixture.

12. A process according to claim 4 comprising (a) contacting the enzyme with the substrate and (b) isolating the L-galactono-1,4-lactone or L-galactonic acid from the reaction mixture.

13. A process according to claim 2, wherein the process is conducted for 1 to 120 h at a pH of about 1 to about 9 and at a temperature of about 13° C to about 45° C.

14. A process according to claim 3, wherein the process is conducted for 1 to 120 h at a pH of about 1 to about 9 and at a temperature of about 13° C. to about 45° C.

15. A process according to claim 4, wherein the process is conducted for 1 to 120 h at a pH of about 1 to about 9 and at a temperature of about 13° C. to about 45° C.

16. A process according to claim 5, wherein the process is conducted for 1 to 120 h at a pH of about 1 to about 9 and at a temperature of about 13° C. to about 45° C.

17. A process according to claim 11, wherein the process is conducted for 1 to 120 h at a pH of about 1 to about 9 and at a temperature of about 13° C. to about 45° C.

18. A process according to claim 12, wherein the process is conducted for 1 to 120 h at a pH of about 1 to about 9 and at a temperature of about 13° C. to about 45° C.
